(19) United States
(12) Patent Application Publication
(10) Pub. No.: US 2026/0209081 A1
(43) Pub. Date: Jul. 23, 2026

(54) A UV-GERMICIDAL IRRADIATION SYSTEM FOR GERMICIDAL TREATMENT OF LIQUIDS

(71) Applicant: LYRAS DK APS, Aalborg (DK)

(72) Inventors: Kasper Dalager VESTDAM, Aalborg (DK); Thomas Yssing MICHAELSEN, Aalborg (DK); Mathias Kræmmergaard KRISTENSEN, Aalborg (DK)

(73) Assignee: LYRAS DK APS, Aalborg (DK)

(21) Appl. No.: 19/157,797

(22) PCT Filed: Feb. 22, 2024
(86) PCT No.: PCT/EP2024/054492

§ 371 Date: Aug. 19, 2025

(30) Foreign Application Priority Data
Feb. 24, 2023 (EP) 23158392.3

(51) Int. Cl. C02F 1/32; A23B 2/53; A23B 11/16; A23B 70/50; A23B 85/10; A23B 90/10

(52) U.S. Cl. CPC C02F 1/325; A23B 2/53; A23B 11/164; A23B 70/50; A23B 85/10; A23B 90/10; C02F 2201/3227; C02F 2301/026; C02F 2303/04

(57) ABSTRACT

A UV-germicidal irradiation system for treatment of liquids, such as opaque liquids, wherein the UV-germicidal treatment system includes a spiral-shaped tube creating a coiled fluidic pathway extending along a longitudinal axis and one or more light sources configured for illuminating the spiral-shaped tube.

### TECHNICAL FIELD

The invention relates to a UV-germicidal irradiation system for germicidal treatment of liquids, which enables a germicidal treatment of liquids utilizing UV-C light, primarily in the wavelength between 180 nm to 300 nm. The invention further relates to a system capable of germicidal treatment of highly opaque liquids. Highly opaque liquids is also to be understood as liquids with low transparency.

### BACKGROUND ART

UV-germicidal irradiation systems have previous been used for treatment of liquids, such as liquid food products. One example of such instrument can e.g. be found in US 2002/096648, which discloses a system for irradiating ultraviolet (UV) light into a fluid medium. An irradiation chamber is connected to an inlet and an outlet which allows the medium to flow through the chamber while being exposed to UV light. Another example of such an instrument is disclosed in US 2004/248076, which shows an apparatus and process for sterilization of liquid media by means of UV irradiation and short-time heat treatment.

However, there is still a need within the field for optimizing the killing of bacteria and viruses (i.e. pasteurization or sterilization) of liquid products while avoiding or lowering the damages to other components within such liquid products, as such damages may result in change of taste or an in another way inferior liquid product.

### SUMMARY

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context, e.g. a composition described herein as comprising a particular element should be understood as also describing a composition consisting of that element, unless otherwise stated or clearly contradicted by context. It will be further understood that the terms “comprises,” “comprising,” “includes” and/or “including,” when used in this specification, specify the presence of stated features, integers, steps, operations, elements, and/or components, but do not preclude the presence or addition of one or more other features, integers, steps, operations, elements, components, and/or groups thereof.

The present invention solves among other the above object by providing a UV-germicidal irradiation system comprising a coiled or spiral-shaped section that is radiated with UV light. This section is defined to be within a very confined geometry, which has been found to be the optimal geometry to have the optimal conditions for killing the bacteria and viruses in the liquid to be treated while similarly having the optimal conditions for minimizing or avoiding damages to other components within the liquid being treated.

Hence, disclosed herein in a first aspect is a UV-germicidal irradiation system for treatment of liquids, such as opaque liquids. The UV-germicidal treatment system comprises at least one tube creating a fluidic pathway, the at least one tube having an inlet section, a coil section, and an outlet section, wherein the coil section is a spiral-shaped tube creating a coiled fluidic pathway extending along a longitudinal axis from the inlet section to the outlet section. The spiral-shaped tube is made of a polymeric material being at least ultraviolet light transparent at wavelengths between 180 and 315 nm. The UV-germicidal treatment system further comprises one or more light sources configured for illuminating the coiled fluidic pathway of the spiral-shaped tube, wherein the one or more light sources emit light with at least one wavelength within a wavelength range between 180 nm and 315 nm. The spiral-shaped tube of the UV-germicidal system has an internal diameter (ID) between 2 mm and 20 mm, a wall thickness (WT) between 5% and 15% of the internal diameter (ID), and an inner coil diameter (ICD) equal to or less than 80 mm. The spiral-shaped tube further has a radius of curvature (RC) between 2.0 and 5.0 times the internal diameter (ID). Further, the spiral-shaped tube is geometrically configured for creating a flow in the coiled fluidic pathway having a Dean number between 300 and 30000. The radius of curvature (RC) is calculated according to EQ. 1:

$$RC = ( ( ICD + ID ) / 2 ) + WT \qquad ( EQ . 1 )$$

The spiral-shaped tube of the present disclosure is in the first aspect made of a polymeric material being at least ultraviolet light transparent at wavelengths between 180 and 315 nm. By a polymeric material being at least ultraviolet light transparent at wavelengths between 180 and 315 nm is meant that at least 5% of UV-light at the given wavelengths are allowed to pass through 0.1 mm of said material. Hence, in one or more embodiments, the polymeric material has an ultraviolet transmittance (UVT) of at least 5%, such as at least 10%, such as at least 15% at wavelengths between 180 and 315 nm.

The polymeric material may be selected from amorphous fluoropolymer (AF), polypropylene (PP), fluorinated ethylene propylene (FEP), polyethylene (PE), polytetrafluoroethylene (PTFE), perfluoroalkoxy alkanes (PFA), polyvinylidene fluoride (PVDF), nylon, rubber, latex, acrylic glass, polycarbonate (PC), polystyrene, acrylonitrile butadiene styrene (ABS), a quartz material, or combinations thereof.

Disclosed herein in a second aspect is a UV-germicidal irradiation system for treatment of liquids, such as opaque liquids. The UV-germicidal treatment system comprises at least one tube creating a fluidic pathway, the at least one tube having an inlet section, a coil section, and an outlet section, wherein the coil section is a spiral-shaped tube creating a coiled fluidic pathway extending along a longitudinal axis from the inlet section to the outlet section. The spiral-shaped tube is made of a quartz material being at least ultraviolet light transparent at wavelengths between 180 and 315 nm. The UV-germicidal treatment system further comprises one or more light sources configured for illuminating the coiled fluidic pathway of the spiral-shaped tube, wherein the one or more light sources emit light with at least one wavelength within a wavelength range between 180 nm and 315 nm. The spiral-shaped tube of the UV-germicidal system has an internal diameter (ID) between 2 mm and 20 mm, a wall thickness (WT) between 5% and 15% of the internal diameter (ID), and an inner coil diameter (ICD) equal to or less than 80 mm. The spiral-shaped tube further has a radius of curvature (RC) between 2.0 and 5.0 times the internal diameter (ID). Further, the spiral-shaped tube is geometrically configured for creating a flow in the coiled fluidic pathway having a Dean number between 300 and 30000. The radius of curvature (RC) is calculated according to EQ. 1:

$$RC = ( ( ICD + ID ) / 2 ) + WT \qquad ( EQ . 1 )$$

The spiral-shaped tube of the present disclosure is in the second aspect made of a quartz material being at least ultraviolet light transparent at wavelengths between 180 and 315 nm. By a quartz material being at least ultraviolet light transparent at wavelengths between 180 and 315 nm is meant that at least 5% of UV-light at the given wavelengths are allowed to pass through 0.1 mm of said material. Hence, in one or more embodiments, the quartz material has an ultraviolet transmittance (UVT) of at least 5%, such as at least 10%, such as at least 15% at wavelengths between 180 and 315 nm.

The spiral-shaped tube of the UV-germicidal system according to the first aspect and the second aspect is also defined by the internal diameter (ID), the wall thickness (WT), and the inner coil diameter (ICD). The internal diameter (ID) is the outer tube diameter (OTD) minus two times the wall thickness (WT) calculated using EQ. 2:

$$ID = OTD - ( 2 \times WT ) \qquad ( EQ . 2 )$$

The outer tube diameter (OTD) is the diameter of the tube measured from outside the wall of said tube to the adjacent outside the wall of the tube. The outer tube diameter (OTD) will always be larger than the internal diameter (ID). The outer tube diameter can also be determined by measuring the circumference of said tube and divide the circumference with pi (π). The wall thickness (WT) is half of the outer tube diameter (OTD) minus the internal diameter (ID) calculated using EQ. 3:

$$WT = ( O TD - ID ) / 2 \qquad ( EQ . 3 )$$

The inner coil diameter (ICD) is the inside width of a coil created by the at least one spiral-shaped tube along the longitudinal axis of the elongated support structure. The inner coil diameter is measured as the distance from inner side to inner side of the spiral-shaped tube after a half turn/coil of the spiral-shaped tube. That is, the coil diameter is the inner width of a coil created by the spiral-shaped tube.

The spiral-shaped tube of the UV-germicidal system is normally uniform in the sense that the variations in the internal diameter (ID), the wall thickness (WT), the inner coil diameter (ICD), and the outer tube diameter (OTD) over the length of the spiral-shaped tube are very small. When reference is made to either the internal diameter (ID), the wall thickness (WT), the inner coil diameter (ICD), and the outer tube diameter (OTD), it is to the average value of the internal diameter (ID), the wall thickness (WT), the inner coil diameter (ICD), and the outer tube diameter (OTD), respectively, over the length of the spiral-shaped tube.

The internal diameter (ID) of the spiral-shaped tube has a minimum internal diameter (IDmin) representing the smallest internal diameter over the length of the spiral-shaped tube and a maximum internal diameter (IDmax) representing the largest internal diameter over the length of the spiral-shaped tube. The minimum internal diameter (IDmin) is normally smaller than the maximum internal diameter (IDmax) by at the most 10%, such as at the most 8%, such as at the most 6%, such as at the most 4%, or such as at the most 2%.

The outer tube diameter (OTD) of the spiral-shaped tube has a minimum outer tube diameter (OTDmin) representing the smallest outer tube diameter over the length of the spiral-shaped tube and a maximum outer tube diameter (OTDmax) representing the largest outer tube diameter over the length of the spiral-shaped tube. The minimum outer tube diameter (OTDmin) is normally smaller than the maximum outer tube diameter (OTDmax) by at the most 10%, such as at the most 8%, such as at the most 6%, such as at the most 4%, or such as at the most 2%.

The wall thickness (WT) of the spiral-shaped tube has a minimum wall thickness (WTmin) representing the smallest wall thickness over the length of the spiral-shaped tube and a maximum wall thickness (WTmax) representing the largest wall thickness over the length of the spiral-shaped tube.

In an example, the wall thickness (WT) of the spiral-shaped tube is 0.35 mm with a variation of +/−0.08 mm. Thus, the maximum wall thickness (WTmax) is in an example 0.43 mm and the minimum wall thickness (WTmin) is 0.27 mm. Thus, the minimum wall thickness (WTmin) is normally smaller than the maximum wall thickness (WTmax) by at the most 50%, such as at the most 40%, such as at the most 30%, such as at the most 20%, such as at the most 10%, or such as at the most 5%.

The inner coil diameter (ICD) of the spiral-shaped tube has a minimum inner coil diameter (ICDmin) representing the smallest inner coil diameter over the length of the spiral-shaped tube and a maximum inner coil diameter (ICDmax) representing the largest inner coil diameter over the length of the spiral-shaped tube. The minimum inner coil diameter (ICDmin) is normally smaller than the maximum inner coil diameter (ICDmax) by at the most 20%, such as at the most 15%, such as at the most 10%, such as at the most 5%, or such as at the most 2%. The inner coil diameter (ICD) of the spiral-shaped tube may change when a liquid runs through the spiral-shaped tube, and may also depend on the pressure of the liquid running through the spiral-shaped tube. The inner coil diameter (ICD) of the spiral-shaped tube is therefore measured when no liquid is in the spiral-shaped tube.

Likewise, the above relative measures of the internal diameter (ID), the wall thickness (WT), and the outer tube diameter (OTD) should be understood as the relative measures at least when no liquid is in the spiral-shaped tube.

Disclosed herein in a third aspect is a use of a UV-germicidal system according to the first aspect or the second aspect for germicidal treatment of liquids, such as opaque liquids.

Liquids as defined herein may refer to both slurries and dissolved solutions without any suspended particles.

The system may also be configured for slurries, and/or fermentation liquids.

One of the advantages of using light radiation as a means for cold pasteurization is that it is a very energy efficient way for partial sterilization. Pasteurization is not only limited to partial sterilization of a substance and especially a liquid at a temperature and for a time period of exposure that destroys objectionable organisms without major chemical alteration of the substance. Pasteurization also covers cold pasteurization which is partial sterilization of a substance and especially a liquid in a process where heat is evaded as the main eradication of objectionable organisms without major chemical alteration of the substance. With evaded is not meant excluded but reduced. The fluidic pathway is normally designed to provide a high surface to volume ratio, increasing the exposure of light energy per unit volume with reduced self-shadowing effects from the opaque liquid being treated. In this manner it is possible to treat opaque liquids using light when the material, creating the fluidic pathway, is at least partly transparent to the radiation of light. The fluidic pathway is normally designed to provide a high surface to volume ratio, increasing the exposure of light energy per unit volume with reduced self-shadowing effects from the liquid being treated. In this manner it is possible to treat opaque liquids using light when the material, creating the fluidic pathway is at least partly transparent to the radiation of light.

By using the at least one tube with the very confined geometry as defined in the first aspect or the second aspect for creating a fluidic pathway in a UV-germicidal irradiation system for treatment of liquids, such as opaque liquids, an improved geometric balance is obtained, which provide optimal conditions for killing the bacteria and viruses in the liquid to be treated while minimizing or avoiding damages to other components within the liquid being treated.

Disclosed herein in a fourth aspect is a use of a UV-germicidal system according to the first aspect or the second aspect for germicidal treatment of industrial fermentation liquids and/or fermentation broth.

Disclosed herein in a fifth aspect is a use of a UV-germicidal system according to the first aspect or the second aspect for killing microorganisms in liquids, such as bacteria, mold, spores, protozoa or virus.

With killing is meant reducing the amount of active or living microorganisms. Microorganisms found in liquid food products may be present due to contamination during the process of said liquid food product. Common bacteria contamination of e.g. dairy products may be e.g., Lactobacillus casei, Escherichia coli, Listeria monocytogenes, Salmonella spp., Mycobacterium avium subspecies paratuberculosis (MAP), Staphylococcus aureus, or Streptococcus spp.

Lastly, disclosed herein in a sixth aspect is a method of germicidal treatment of an industrial fermentation liquid, the method comprising the steps of: providing an industrial fermentation liquid to be treated; passing the industrial fermentation liquid to be treated through at least one tube in a UV-germicidal irradiation system according to the first aspect or the second aspect at a predefined flow speed, while illuminating the coiled fluidic pathway of the spiral-shaped tube with at least one wavelength within a wavelength range between 180 nm and 315 nm from the one or more light sources at a predefined power-output to obtain a germicidal treated fermentation liquid; and collecting the germicidal treated fermentation liquid.

Germicidal treatment is not only limited to partial sterilization of a substance and especially a liquid at a temperature and for a time period of exposure that destroys objectionable organisms without major chemical alteration of the substance, but also covers cold pasteurization which is partial sterilization of a substance and especially a liquid in a process where heat is evaded as the main eradication of objectionable organisms without major chemical alteration of the substance. With evaded is not meant excluded but reduced. The present invention discloses that one of the advantages of using light radiation as a means for germicidal treatment is that it is a very energy efficient way for partial sterilization.

Unless otherwise defined, all terms used herein (including technical and scientific terms) have the same meaning as commonly understood by those skilled in the art to which this invention pertains. It will be further understood that terms, such as those defined in commonly used dictionaries, should be interpreted as having a meaning that is consistent with their meaning in the context of the relevant art and will not be interpreted in an idealized or overly formal sense unless expressly so defined in the present specification.

The present disclosure will become apparent from the detailed description given below. The detailed description and specific examples disclose preferred embodiments of the disclosure by way of illustration only. Those skilled in the art understand from guidance in the detailed description that changes and modifications may be made within the scope of the disclosure.

### BRIEF DESCRIPTION OF THE DRAWINGS

FIG. 1 shows a coil cross section of a tube within the UV-germicidal irradiation system of the spiral-shaped tube.

FIG. 2 shows a coil cross section of the spiral-shaped tube.

FIG. 3 shows a spiral-shaped tube in a side view.

FIG. 4 shows a UV-germicidal irradiation system in a perspective view.

FIG. 5 shows a graph showing two functions for two different coil types, both falling within the present invention.

FIG. 6 shows a graph showing three functions for three different coil types, two falling within the present invention and one falling outside.

### DETAILED DESCRIPTION

As used herein, the singular forms “a,” “an,” and “the” are intended to include the plural forms, including “at least one,” unless the content clearly indicates otherwise. “At least one” is not to be construed as limiting “a” or “an.”

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The present invention is focusing on solving the problem of providing a system, which is optimized for UV-germicidal treatment, i.e. killing bacteria and viruses in a liquid product. However, when UV treating liquid product there is a risk of damaging some of the components in such product with the UV light used for the germicidal treatment. The present invention is therefore also focusing on avoiding or minimizing this risk, as this damaging or altering of component in the product may result in a change in taste of said product or in a loss of nutrients etc., hereby creating an inferior product.

The present invention has nicely solved this problem by utilizing a coiled or spiral-shaped section that is radiated with UV light within the system. This section is defined to be within a very confined geometry, which among other is defined by a radius of curvature (RC) corelated with the internal diameter (ID), the wall thickness (WT), and the inner coil diameter (ICD). The geometry is further configured to create a flow created within the spiral-shaped tube within a certain range in regards to the Dean number.

When UV-treating liquids, especially liquids that are prone to damage by the UV light, it may be important to prevent or reduce the amount of light at wavelength above the UVC spectrum being emitted onto the liquid. Light sources used at present in UV-germicidal systems do not utilise a lamp source, which provides just a single wavelength, i.e. where 100% of the light is emitted at a single wavelength. Therefore, though light sources are normally referred to as having a specific wavelength, the specific light source wavelength is normally representing the peak wavelength or the wavelength having the highest intensity, where the light source further also emits light having wavelengths neighbouring the peak wavelength. Thus, the light sources used at present normally emit light in a spectrum of wavelength with a specific wavelength being the wavelength having the highest light intensity. For this reason, it is important to minimize the wavelengths having a negative impact on certain liquids being treated with the UV light.

Looking at a lamp profile of a commonly used low pressure germicidal UV lamp known to a skilled person, which is often referred to within the filed as emitting “a single wavelength” and/or emitting “monochromatic light” it can be seen that the lamp emits several other wavelengths, which are undesirable/needless when treating liquids. Looking at another lamp of a commonly used medium pressure germicidal UV lamp, which is often referred to within the field as emitting multiple wavelengths, it can also be seen that the lamp emits several wavelengths above 315 nm, which can be undesirable/needless. By looking at these lamp profiles, which is light sources commonly used in UV-germicidal systems, it is evident that there are more than one single wavelength emitted when using a UV lamp. It is also evident that the lamp sources emit wavelengths above the UV spectrum. The peaks above 315 nm are not commonly mentioned, as they serve no positive purpose when using UV light treating water etc.; however, they are still present in all UV light sources presently used. Wavelengths above 315 nm can have a negative impact when treating other liquids than water. The present invention solves this problem by finding the sweet spot where the dose of light given within the germicidal wavelength is optimized, i.e. sufficient dose to treat the liquid, while the dose of light given in the undesired wavelength is minimized, i.e. insufficient dose in said wavelength, so the liquid is not damaged. This also means that the present invention discloses a system, where the tube geometry ensures that liquid being treated in the system is only present in the spiral-shaped tube and being treated for the exact amount of time to ensure a sufficient germicidal treatment, while not staying in the tube for a period long enough to overexpose the liquid to the negatively impacting wavelengths.

Further, by designing the fluidic pathway in this way, it is ensured that the fluidic pathway provides a high surface to volume ratio, increasing the exposure of light energy per unit volume with reduced self-shadowing effects from the liquid being treated, especially if treating opaque liquids. In this manner it is possible to treat opaque liquids using UV light when the material, creating the fluidic pathway, is transparent to the radiation of the UV light.

In one or more embodiments, the spiral-shaped tube has a compressed length (CL) equal to or less than 2500 mm, such as equal to or less than 1250 mm, such as equal to or less than 1150 mm, or such as equal to or less than 1050 mm.

The compressed length (CL) is the length of the spiral-shaped tube creating the coiled fluidic pathway measured along the longitudinal axis of the spiral-shaped tube without pulling or pressing on the spiral-shaped tube. The compressed length (CL) may also be defined as the longitudinal linear length (LL) between the inlet section to the outlet section corresponding to the coil section.

In one or more embodiments, the UV-germicidal irradiation system is configured for creating a flow in the coiled fluidic pathway having a flow speed of the liquid being treated between 0.5 m/s and 10 m/s, such as between 1.0 m/s and 10 m/s, such as between 1.5 m/s and 10 m/s, such as between 1.5 m/s and 8 m/s, such as between 1.5 m/s and 6 m/s, or such as between 3 m/s to 6 m/s.

Flow velocity is the vector field that is used to describe fluid motion in a mathematical manner. The entire length of the flow velocity is referred to as the flow speed. Flow velocity in fluids is the vector field that provides the velocity of fluids at a certain time and position. Flow velocity is also known as macroscopic velocity. In this application, the flow speed is defined as the mass flow divided by fluid density and internal cross-sectional area of the spiral-shaped tube creating a coiled fluidic pathway.

In one or more embodiments, the spiral-shaped tube has a pitch between 1 time the outer tube diameter (OTD) and 4 times the outer tube diameter (OTD), such as between 1 time the OTD and 3 times the OTD, or such as between 1 time the OTD and 2 times the OTD.

The pitch is the distance from centre to centre of the spiral-shaped tube after one turn/coil of the spiral-shaped tube. The pitch may be defined based on the outer tube diameter. If the pitch is 1-2 times the OTD, it is preferred that one spiral-shaped tube is extending along the longitudinal axis from the inlet section to the outlet section. However, if the pitch is 3-4 times the OTD, the spiral-shaped tube may be what is called a “short” coil, which is then only roughly half the compressed length (CL) from inlet section to outlet section along the longitudinal axis. With a “short” coil it may be preferred to have two spiral-shaped tubes extending along the longitudinal axis from the inlet section to the outlet section, where these two spiral-shaped tubes are intercalated/intertwined with each other. In one or more embodiments, the spiral-shaped tube has a pitch between 6 and 48 mm, such as between 6 and 36 mm, or such as between 6 and 24 mm. In one or more embodiments, the spiral-shaped tube has a pitch between 12 and 48 mm, such as between 12 and 36 mm, or such as between 12 and 24 mm. In one or more embodiments, the spiral-shaped tube has a pitch between 6 and 24 mm, such as between 6 and 18 mm, or such as between 6 and 12 mm.

In one or more embodiments, the spiral-shaped tube has a coil angle between 0.5° to 45°, such as between 1.5° to 35°, such as between 2.5° to 25°, or such as between 2.5° to 20°.

The coil angle is measured between the spiral-shaped tube and a straight direction extending radially from the longitudinal axis of the spiral-shaped tube.

In one or more embodiments, the coiled fluidic pathway from the inlet section to the outlet section is at least 1 m long, such as at least 2 m long, such as at least 5 m long, or such as at least 10 m long.

The length of the coiled fluidic pathway may also be termed the extension- or free length of the spiral-shaped tube. The length of the coiled fluidic pathway is the total length the coiled fluidic pathway of the spiral-shaped tube. The total length of the coiled fluidic pathway is equal to the total distances one liquid food product unit has to pass through the spiral-shaped tube. In one or more embodiments, the coiled fluidic pathway from the inlet section to the outlet section is between 1 m and 100 m long, such as between 1 m and 50 m long, such as between 1 m and 25 m long, such as between 1 m and 20 m long.

In one or more embodiments, the spiral-shaped tube has an outer tube diameter (OTD) between 2.0 mm and 26.0 mm, such as between 3.0 mm and 26.0 mm, such as between 4.0 mm and 26.0 mm, such as between 5.0 mm and 26.0 mm, such as between 6.0 mm and 26.0 mm, such as between 6.0 mm and 20.0 mm, such as between 6.0 mm and 17.0 mm, such as between 6.0 mm and 14.0 mm, or such as between 6.0 mm and 12.0 mm. The spiral-shaped tube of the UV-germicidal system is normally uniform in the sense that the variations in the outer tube diameter (OTD) over the length of the spiral-shaped tube is small. When reference is made to the outer tube diameter (OTD), it is to the average value of the outer tube diameter (OTD) over the length of the spiral-shaped tube.

The outer tube diameter (OTD) of the spiral-shaped tube has a minimum outer tube diameter (OTDmin) representing the smallest internal diameter over the length of the spiral-shaped tube and a maximum outer tube diameter (OTDmax) representing the largest internal diameter over the length of the spiral-shaped tube. The minimum outer tube diameter (OTDmin) is normally smaller than the maximum outer tube diameter (OTDmax) by at the most 10%, such as at the most 8%, such as at the most 6%, such as at the most 4%, or such as at the most 2%.

In one or more embodiments, the spiral-shaped tube has an internal diameter (ID) between 2 mm and 18 mm, such between 2 mm and 16 mm, such between 2 mm and 14 mm, such between 2 mm and 12 mm, such as between 4 mm and 12 mm, such as between 5 mm and 12 mm, or such as between 5 mm and 10 mm. The spiral-shaped tube of the UV-germicidal system is normally uniform in the sense that the variations in the internal diameter (ID) over the length of the spiral-shaped tube is small. When reference is made to the internal diameter (ID), it is to the average value of the internal diameter (ID) over the length of the spiral-shaped tube.

The internal diameter (ID) of the spiral-shaped tube has a minimum internal diameter (IDmin) representing the smallest internal diameter over the length of the spiral-shaped tube and a maximum internal diameter (IDmax) representing the largest internal diameter over the length of the spiral-shaped tube. The minimum internal diameter (IDmin) is normally smaller than the maximum internal diameter (IDmax) by at the most 10%, such as at the most 8%, such as at the most 6%, such as at the most 4%, or such as at the most 2%.

In one or more embodiments, the spiral-shaped tube has a wall thickness (WT) between 5% and 15% of the internal diameter (ID), such as between 5% and 14%, such as between 5% and 12%, or such as between 5% and 10%. The spiral-shaped tube of the UV-germicidal system is normally uniform in the sense that the variations in the wall thickness (WT) over the length of the spiral-shaped tube is small. When reference is made to the wall thickness (WT), it is to the average value of the wall thickness (WT) over the length of the spiral-shaped tube.

The wall thickness (WT) of the spiral-shaped tube has a minimum wall thickness (WTmin) representing the smallest internal diameter over the length of the spiral-shaped tube and a maximum wall thickness (WTmax) representing the largest internal diameter over the length of the spiral-shaped tube. The minimum wall thickness (WTmin) is normally smaller than the maximum wall thickness (WTmax) by at the most 50%, such as at the most 40%, such as at the most 30%, such as at the most 20%, such as at the most 10%, or such as at the most 5%.

In one or more embodiments, the inner coil diameter (ICD) is at least 10 mm. In one or more embodiments, the spiral-shaped tube has an inner coil diameter (ICD) equal to or less than 70 mm, such as equal to or less than 60 mm, such as equal to or less than 50 mm, or such as between 10 mm and 50 mm. The spiral-shaped tube of the UV-germicidal system is normally uniform in the sense that the variations in the inner coil diameter (ICD) over the length of the spiral-shaped tube is small. When reference is made to the inner coil diameter (ICD), it is to the average value of the inner coil diameter (ICD) over the length of the spiral-shaped tube.

The inner coil diameter (ICD) of the spiral-shaped tube has a minimum inner coil diameter (ICDmin) representing the smallest internal diameter over the length of the spiral-shaped tube and a maximum inner coil diameter (ICDmax) representing the largest internal diameter over the length of the spiral-shaped tube. The minimum inner coil diameter (ICDmin) is normally smaller than the maximum inner coil diameter (ICDmax) by at the most 20%, such as at the most 15%, such as at the most 10%, such as at the most 5%, or such as at the most 2%.

In one or more embodiments, the spiral-shaped tube has a radius of curvature (RC) between 2.6 and 4.6 times the internal diameter (ID).

In one or more embodiments, the spiral-shaped tube is geometrically configured for creating a flow in the coiled fluidic pathway having a Dean number between 300 and 25000, such as between 600 and 25000, such as between 750 and 25000, such as between 1000 and 25000, such as between 1500 and 25000, or such as between 2000 and 25000.

In one or more embodiments, the UV-germicidal irradiation system further comprises at least one elongated support structure, and wherein the spiral-shaped tube is coiled around the elongated support structure along the longitudinal axis of the spiral-shaped tube.

In one or more embodiments, one or more light sources illuminate at least 50% of the coiled fluidic pathway of the spiral-shaped tube, such as at least 60%, such as at least 70%, such as at least 75%, such as at least 80%, such as at least 90%, or such as at least 95% of the coiled fluidic pathway of the spiral-shaped tube.

In one or more embodiments, the liquids are liquids having an ultraviolet transmittance (UVT) between 1% and 95%, such as between 5% and 90%, wherein UVT is measured at 254 nm light and in a 0.1 mm path length. To measure ultraviolet transmittance (UVT), ultraviolet light at 254 nm is passed through a quartz cell containing the sample liquid with a liquid column thickness of 0.1 mm. The UVT is a measure of the amount of light passing through the quartz cell, such that the more opaque or cloudy the liquid is the lower the UVT will be.

In one or more embodiments, the liquids are liquid food products.

Liquid food products are products that can be used or prepared for use as food for human and/or animal consumption. It can also be products that can be used as an ingredient in said food or used in a process for preparing said food.

In one or more embodiments, the liquids are industrial fermentation liquids and/or fermentation broth, such as enzymatic liquids.

In one or more embodiments, the liquids are selected from fermentation liquid, fermentation broth, raw milk, milk, whey, juice, coffee, tea, soya, soylent, soda or soft drink, broth, soup, beer, smoothies, protein shake, liquid meal-replacement, cream, wine, mayonnaise, ketchup, syrup, honey, processing water, blood or blood plasma, cider, brine solution, human milk, ice tea, yogurt, spirits, cocktails, mocktails, sugary slurries, lactoferrin, smoothie, chocolate milk, plant-based milk, soya drink, ice tea, egg, bactofugat, or combinations thereof.

In one or more embodiments, the UV-germicidal system further comprises one or more filters, wherein the one or more filters are positioned between the one or more light sources and the coiled fluidic pathway of the spiral shaped tube.

In one or more embodiments, the one or more filters are positioned such that light emitted from the one or more light sources passes through the one or more filters prior to reaching the coiled fluidic pathway of the spiral-shaped tube.

In one or more embodiments, the one or more filters attenuate light with a wavelength between 310 nm and 460 nm, such as between 290 nm and 460 nm, such as between 290 nm and 490 nm, by a factor of at least 10, such as a factor of at least 20, such as a factor of at least 50, such as a factor of at least 100, such as a factor of at least 500, or such as a factor of at least 1000.

In one or more embodiments, the one or more filters are selected from bandpass filters, notch filters, or a combination of both.

In one or more embodiments, the at least one elongated support structure is selected from one or more rods, a pillar, a cylindric mesh, and/or one or more hollow rods.

In one or more embodiments, the polymeric material is selected from amorphous fluoropolymer (AF), polypropylene (PP), fluorinated ethylene propylene (FEP), polyethylene (PE), polytetrafluoroethylene (PTFE), perfluoroalkoxy alkanes (PFA), polyvinylidene fluoride (PVDF), nylon, rubber, latex, acrylic glass, polycarbonate (PC), polystyrene, acrylonitrile butadiene styrene (ABS), a quartz material, or combinations thereof.

In one or more embodiments, the inlet section and the outlet section are designed such that the liquid enters and exits the coiled section axially and/or radially.

In one or more embodiments, the inlet section and the outlet section are designed such that the liquid enters and exits the coiled section axially. In one or more embodiments, the inlet section and the outlet section is designed such that the liquid enters and exits the coiled section radially.

In one or more embodiments, the inlet section and the outlet section are designed such that the liquid flows overall vertically through coiled fluidic pathway when observing from an inlet end of the coiled section to an outlet end of the coiled section.

In one or more embodiments, the inlet section and the outlet section are designed such that the liquid flows overall horizontally through the coiled fluidic pathway when observing from an inlet end of the coiled section to an outlet end of the coiled section.

By “designed such that the liquid flows overall horizontally through the coiled fluidic pathway when observing from an inlet end of the coiled section to an outlet end of the coiled section” is meant that the liquid flows overall horizontally through the coil section, i.e. the spiral-shaped tube, when observing over the length of the spiral-shaped tube. This means that the liquid will enter the coil section horizontally, flow through the spiral-shaped tube, and exit the coil section horizontally, hereby giving an overall horizontal flow.

In one or more embodiments, the one or more light sources are selected from a mercury-vapor lamp, xenon lamp, light emitting diode (LED) or pulsed LED.

A mercury-vapor lamp is a gas discharge lamp that uses an electric arc through vaporized mercury to produce light. The arc discharge may be confined to a small fused quartz arc tube.

A light emitting diode (LED) is a two-lead semiconductor light source. It is a p-n junction diode that emits light when activated. When a suitable voltage is applied to the leads, electrons are able to recombine with electron holes within the device, releasing energy in the form of photons. This effect is called electroluminescence, and the color of the light (corresponding to the energy of the photon) is determined by the energy band gap of the semiconductor. LEDs are typically small (less than 1 mm) and integrated optical components may be used to shape the radiation pattern.

A xenon lamp, or a xenon arc lamp, is a specialized type of gas discharge lamp, an electric light that produces light by passing electricity through ionized xenon gas at high pressure. It produces a bright white light that closely mimics natural sunlight. A special kind of xenon lamp is used in automobiles. These are actually metal-halide lamps, where a xenon arc is only used during start-up.

In one or more embodiments, the one or more light sources are a low pressure germicidal lamp, such as a low-pressure mercury-vapor lamp or a medium pressure germicidal lamp, such as a medium-pressure mercury-vapor lamp.

In one or more embodiments, the one or more light sources are a medium pressure germicidal lamp, such as a medium-pressure mercury-vapor lamp.

In one or more embodiments, the UV-germicidal irradiation system is configured for inactivating or reducing a biological contaminant by an order of at least at least 1-Log₁₀, such as at least 2-Log₁₀, such as at least 3-Log₁₀, such as at least 4-Log₁₀, such as at least 5-Log₁₀, such as at least 6-Log₁₀.

In one or more embodiments, the biological contaminant is selected from Campylobacter jejuni, Shigella, Coxiella burnetii, Escherichia coli, Listeria monocytogenes, Mycobacterium bovis, Mycobacterium tuberculosis, Mycobacterium paratuberculosis, Salmonella spp., Yersinia enterocolitica, Brucella spp., Staphylococcus spp., Lactobacillus casei, Mycobacterium avium subspecies, Staphylococcus aureus, Streptococcus spp., Enterococcus spp., or Enterobacter spp.

In one or more embodiments, the spiral-shaped tube is made of a polymeric material being at least ultraviolet light transparent at wavelengths between 250 nm and 260 nm, such as between 245 nm and 265 nm, such as between 240 nm and 270 nm, such as between 230 nm and 280 nm, such as between 220 nm and 290 nm, such as between 210 nm and 300 nm, such as between 200 nm and 310 nm, or such as between 180 nm and 315 nm.

In one or more embodiments, the one or more light sources emit light with at least one wavelength within a wavelength range between 180 nm and 300 nm, such as between 200 nm and 300 nm, such as between 220 nm and 300 nm, such as between 240 nm and 280 nm, such as between 250 nm and 260 nm.

In one or more embodiments, the use for germicidal treatment is for germicidal treatment of liquids having an ultraviolet transmittance (UVT) between 5% and 90%, wherein UVT is measured at 254 nm light and 0.1 mm path length.

In one or more embodiments, the fermentation liquid to be treated is a liquid having an ultraviolet transmittance (UVT) between 5% and 90%, wherein UVT is measured at 254 nm light and 0.1 mm path length.

In one or more embodiments, the method further comprises dimensioning the UV-germicidal irradiation system prior to passing the industrial fermentation liquid to be treated through the at least one tube in the UV-germicidal irradiation system.

In one or more embodiments, the predefined flow speed is a flow speed between 0.5 m/s and 10 m/s, such as 1.0 m/s and 10 m/s, such as 1.5 m/s and 10 m/s, such as 1.5 m/s and 8 m/s, such as 1.5 m/s and 6 m/s, or such as 3 m/s to 6 m/s.

In one or more embodiments, the predefined power-output is a power-output between 100 and 1200 W/m².

### DESCRIPTION OF DRAWINGS

Exemplary examples will now be described more fully hereinafter with reference to the accompanying drawings. In this regard, the present examples may have different forms and should not be construed as being limited to the descriptions set forth herein. Accordingly, the examples are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

In the drawings, thicknesses of a plurality of layers and areas are illustrated in an enlarged manner for clarity and ease of description thereof. When a layer, area, element, or plate is referred to as being “on” another layer, area, element, or plate, it may be directly on the other layer, area, element, or plate, or intervening layers, areas, elements, or plates may be present therebetween. Conversely, when a layer, area, element, or plate is referred to as being “directly on” another layer, area, element, or plate, there are no intervening layers, areas, elements, or plates therebetween. Further when a layer, area, element, or plate is referred to as being “below” another layer, area, element, or plate, it may be directly below the other layer, area, element, or plate, or intervening layers, areas, elements, or plates may be present therebetween. Conversely, when a layer, area, element, or plate is referred to as being “directly below” another layer, area, element, or plate, there are no intervening layers, areas, elements, or plates therebetween.

The spatially relative terms “lower” or “bottom” and “upper” or “top”, “below”, “beneath”, “less”, “above”, and the like, may be used herein for ease of description to describe the relationship between one element or component and another element or component as illustrated in the drawings. It will be understood that the spatially relative terms are intended to encompass different orientations of the device in use or operation, in addition to the orientation depicted in the drawings. For example, in the case where a device illustrated in the drawings is turned over, elements described as being on the “lower” side of other elements, or “below” or “beneath” another element would then be oriented on “upper” sides of the other elements, or “above” another element. Accordingly, the illustrative term “below” or “beneath” may include both the “lower” and “upper” orientation positions, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below, and thus the spatially relative terms may be interpreted differently depending on the orientations described.

Throughout the specification, when an element is referred to as being “connected” to another element, the element is “directly connected” to the other element, or “electrically connected” to the other element with one or more intervening elements interposed therebetween.

It will be understood that, although the terms “first,” “second,” “third,” and the like may be used herein to describe various elements, these elements should not be limited by these terms. These terms are only used to distinguish one element from another element. Thus, “a first element” discussed below could be termed “a second element” or “a third element,” and “a second element” and “a third element” may be termed likewise without departing from the teachings herein.

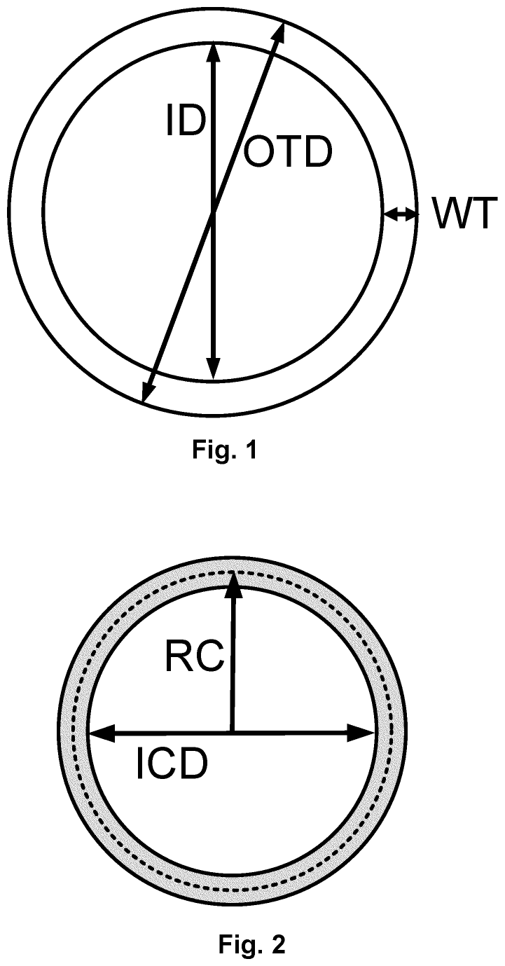

FIG. 1 shows a coil cross section of the tube within the UV-germicidal irradiation system of the spiral-shaped tube. The spiral-shaped tube has an internal diameter (ID), an outer tube diameter (OTD) and a wall thickness (WT) as illustrated in FIG. 1.

The internal diameter (ID) may be between 2 mm and 20 mm, such as between 2 mm and 18 mm, such as between 2 mm and 16 mm, such between 2 mm and 12 mm, such as between 4 mm and 12 mm, such as between 5 mm and 12 mm, or such as between 5 mm and 10 mm.

The spiral-shaped tube may have an outer tube diameter (OTD) between 2.0 mm and 26.0 mm, such as between 3.0 mm and 26.0 mm, such as between 4.0 mm and 26.0 mm, such as between 5.0 mm and 26.0 mm, such as between 6.0 mm and 26.0 mm, such as between 6.0 mm and 20.0 mm, such as between 6.0 mm and 17.0 mm, such as between 6.0 mm and 14.0 mm, or such as between 6.0 mm and 12.0 mm. The outer tube diameter (OTD) will always be larger than the internal diameter (ID).

In one or more examples, the spiral-shaped tube has a wall thickness (WT) between 5% and 15% of the internal diameter (ID), such as between 5% and 14% of the internal diameter (ID), such as between 5% and 12% of the internal diameter (ID), or such as between 5% and 10% of the internal diameter (ID). The wall thickness may also be defined as half of the outer tube diameter minus the inner tube diameter.

The size of the inner diameter (ID), the outer diameter (OTD) and thereby the wall thickness (WT) is a trade-off between the amounts of liquid capable of being treated over a given time versus the exposure of light energy per unit volume/surface area. The larger the tube is, the more liquid can pass over any given time at the same pressure difference. However, the larger the tube is, the smaller (relatively seen) the exposed area may be.

FIG. 2 shows a coil cross section of the spiral-shaped tube, which is substantially perpendicular to the coil cross section as shown in FIG. 1. Here is shown the spiral-shaped tube having an inner coil diameter (ICD). The inner coil diameter is the inner width of a coil created by the spiral-shaped tube.

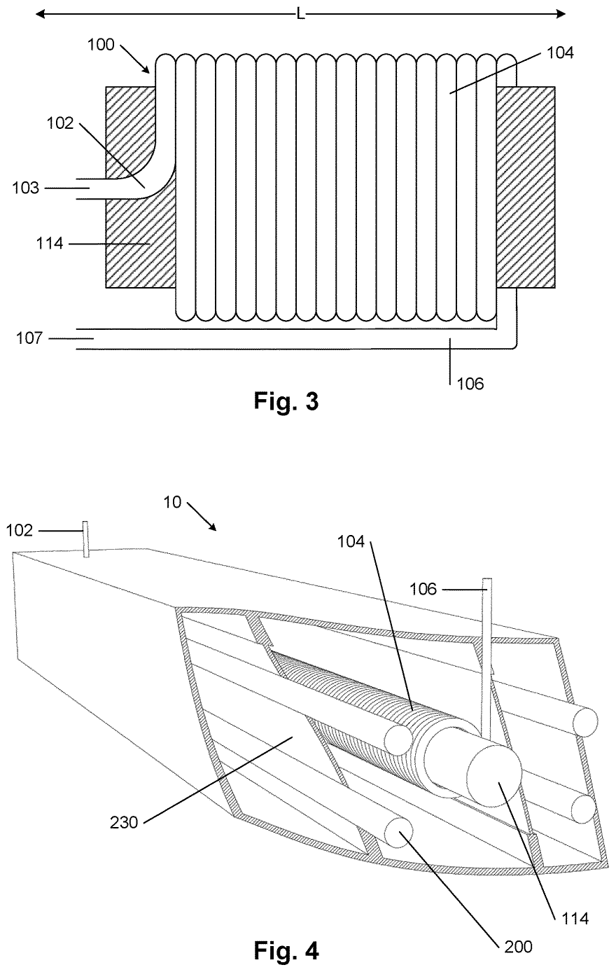

Other aspects and geometries of the coil is shown in FIG. 3 showing the tube 100 in a side view. The spiral-shaped tube 104 may have a pitch defined as the distance from centre to centre of the spiral-shaped tube 104 after one turn/coil of the spiral-shaped tube. In one or more examples, the spiral-shaped tube has a pitch between 1 time the outer tube diameter (OTD) and 4 times the outer tube diameter (OTD), such as between 1 time the outer tube diameter (OTD) and 3 times the outer tube diameter (OTD), or such as between 1 time the outer tube diameter (OTD) and 2 times the outer tube diameter (OTD).

The inlet section 102 comprises a tube inlet 103 through which the liquid enters the tube 100 and the outlet section 106 comprise a tube outlet 107 through which the liquid exits the tube 100.

As exemplified in FIG. 3, the outlet section 106 may extend in parallel with the longitudinal axis L of the coil section 104 such that the tube inlet 103 and the tube outlet 107 are positioned on the side.

The fluid movement of the liquid through the spiral-shaped tube 104 may create a Dean Vortex flow, laminar flow, or turbulent flow in the liquid, where the Dean number is between 300 and 30000.

The spiral-shaped tube 104 may be made of a material selected from the group of amorphous fluoropolymer AF, polypropylene PP, fluorinated ethylene propylene FEP, polyethylene PE, polytetrafluoroethylene PTFE, perfluoroalkoxy alkanes PFA, polyvinylidene fluoride PVDF, nylon, rubber, latex, acrylic glass, polycarbonate PC, polystyrene, acrylonitrile butadiene styrene ABS, or a similar material. The spiral-shaped tube 104 may for example be from amorphous fluoropolymer AF.

The inlet section 102 and the outlet section 106 may be of the same material as the coil section 104. Alternatively, the inlet section 102 and/or the outlet section 106 may be made of a different material than the coil section 104.

The spiral-shaped tube 104 may be made of a polymeric material being at least ultraviolet light transparent at wavelengths between 250 and 260 nm. The ultraviolet light transparency may also cover wavelengths between 240-270 nm, such as between 230 and 280 nm, such as between 210 and 290 nm, such as between 195 and 305 nm, such as between 180 and 315 nm. By a polymeric material being at least ultraviolet light transparent is meant that at least 5% of UV-light at the given wavelengths are allowed to pass through 0.1 mm of said material. In one or more examples, the polymeric material has an ultraviolet transmittance (UVT) of at least 5%, such as at least 10%, such as at least 15% at wavelengths between 250 and 260 nm.

The polymeric material may be amorphous fluoropolymer (AF), polypropylene (PP), fluorinated ethylene propylene (FEP), polyethylene (PE), polytetrafluoroethylene (PTFE), perfluoroalkoxy alkanes (PFA), polyvinylidene fluoride (PVDF), nylon, rubber, latex, acrylic glass, polycarbonate (PC), polystyrene, acrylonitrile butadiene styrene (ABS).

For example, the spiral-shaped tube 104 may be made of a quartz material being at least ultraviolet light transparent at wavelengths between 250 and 260 nm. The ultraviolet light transparency may also cover wavelengths between 240-270 nm, such as between 230 and 280 nm, such as between 210 and 290 nm, such as between 195 and 305 nm, such as between 180 and 315 nm. By a quartz material being at least ultraviolet light transparent is meant that at least 5% of UV-light at the given wavelengths are allowed to pass through 0.1 mm of said material. In one or more examples, the quartz material has an ultraviolet transmittance (UVT) of at least 5%, such as at least 10%, such as at least 15% at wavelengths between 250 and 260 nm.

The system 10 may comprise elongated support structures 114, wherein each spiral-shaped tube 104 is coiled around an elongated support structure along the longitudinal axis L of the spiral-shaped tube 104 as illustrated in FIG. 4. The support structures 114 may be made of or covered by a reflective material, such as a reflective polymeric material or a polished metal.

The system 10 further comprises a number of light sources 200 configured to illuminate the coiled fluidic pathway of the spiral-shaped tube 104. An example of one of the light sources 200 is shown in FIG. 4. The light sources 200 emit light with at least one wavelength within a wavelength range between 180 nm and 315 nm. This wavelength range is also referred to as UV-C light (approximately 100-280 nm) and UV-B light (approximately 280-315 nm). Normally, light within the UV-C wavelength range will be the preferred wavelength range.

The light source 200 may be selected from a mercury-vapor light source, a xenon light source, a light emitting diode LED, or a pulsed LED, or a low pressure germicidal light source, such as a low-pressure mercury-vapor light source.

One or more filters 230 may be positioned between the light sources 200 and the spiral-shaped tube 104 in the plurality of tubes 100. The one or more filters 230 may prevent light above a wavelength of 315 nm from reaching the one or more spiral-shaped tube 104. By “prevent light above a wavelength of 315 nm from reaching the one or more spiral-shaped tube” is meant that the one or more filters attenuates light above 315 nm by at least a factor 10, such as by at least a factor 1.000, such by at least a factor 10.000. The one or more filters may be selected from bandpass filters, notch filters, or a combination of both. Instead of filter, a glass plate may be mounted between the light sources 200 and the spiral-shaped tube 104, or the space may simply be left open.

The UV-germicidal irradiation system 10 is normally configured for inactivating or reducing a biological contaminant by an order of at least 1-Log₁₀, such as at least 2-Log₁₀, such as at least 3-Log₁₀, such as at least 4-Log₁₀, such as at least 5-Log₁₀, such as at least 6-Log₁₀. The biological contaminant may be selected from Campylobacter jejuni, Shigella, Coxiella burnetii, Escherichia coli, Listeria monocytogenes, Mycobacterium bovis, Mycobacterium tuberculosis, Mycobacterium paratuberculosis, Salmonella spp., Yersinia enterocolitica, Brucella spp., Staphylococcus spp., Lactobacillus casei, Mycobacterium avium subspecies, Staphylococcus aureus, Streptococcus spp., Enterococcus spp., or Enterobacter spp.

### REFERENCES

10 UV-germicidal irradiation system
100 tube
102 inlet section
103 tube inlet
104 coil section, spiral-shaped tube
106 outlet section
107 tube outlet
114 elongated support structure
200 light source
230 filter
A-E coil with varying diameter/examples
L longitudinal axis

### EXAMPLES

The present invention is further illustrated by the following examples, which are not to be construed as limiting the scope of protection. The features disclosed in the foregoing description and in the following examples may, both separately or in any combination thereof, be material for realising the invention in diverse forms thereof.

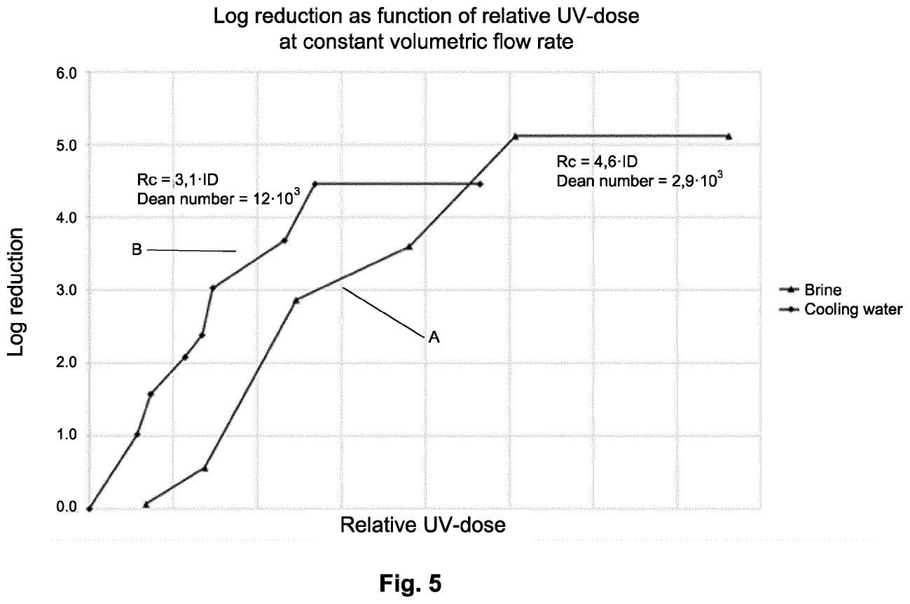

The graph shown in FIG. 5 shows two functions for two different coil types, both falling within the present invention. One is a 5 mm internal diameter coil (A) defined as having a RC equal to 4.6 times the internal diameter, and the other is a 10 mm internal diameter coil (B) defined as having a RC equal to 3.1 times the internal diameter, i.e. both are within the defined geometry. The first coil is being run with brine, while the latter is being run with cooling water. The radius of curvature and dean number is given for both functions in the graph, indicating that the coils fall within the defined range.

The UV-dose values are shown as relative, as they depend on the intensity from the used lamp. The graph shows that the log reduction increases with relative UV-dose, and that it is possible to reach acceptable log reductions with the coils falling within the range.

The results in the graph are found by passing brine and cooling water through the UV unit using two different coils with different flow rates. The UV intensity is varied to vary the relative UV dose and samples are taken for each setting. The brine was analysed for yeast reduction using the standard AFNOR 3M 01/13-07/14 and confirmed by ISO6611. The cooling water was analysed for total plate count (TPC) reduction using the standard ISO 4833-1.

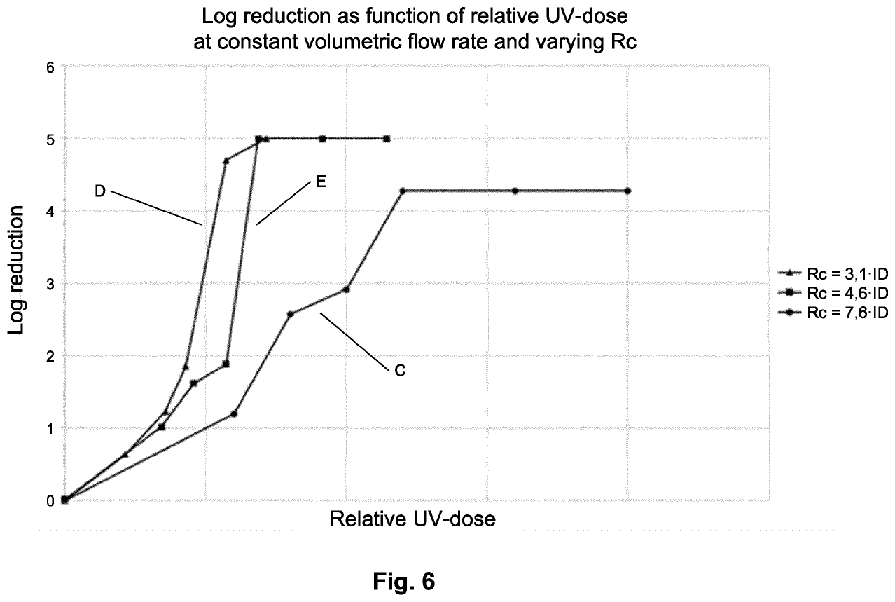

The graph shown in FIG. 6 similarly shows the log reduction at a relative UV-dose, which is normalised by the RC values. The internal diameter of the coil is the same, and the varying factor in the example is the inner coil diameter (ICD). The graph shows that the coil (C), which falls outside the RC range as claimed herein is performing worse (RC equal to 7.6 times ID), than the two coils (D) and (E) within the RC range (RC Equal to 3.1 times ID/4.6 times ID).

In this example, it is the same liquid that is passed through the three different coils and the UV intensity is varied to test the relationship between log reduction and relative UV-dose.

For both sets of data, the UV-dose is relative meaning that it is not a measured value, nor is it an accurate value. The values are however precise and comparable in relation to each other, meaning that the values are not directly comparable to literature data but should only be used as an artificial measurement of the relative performance between coil sizes and flow parameters.

1. A UV-germicidal irradiation system for treatment of liquids, such as opaque liquids, wherein the UV-germicidal treatment system comprises:
at least one tube creating a fluidic pathway, the at least one tube comprising an inlet section, a coil section, and an outlet section, wherein the coil section is a spiral-shaped tube creating a coiled fluidic pathway extending along a longitudinal axis of the spiral-shaped tube from the inlet section to the outlet section, wherein the spiral-shaped tube comprises a polymeric material being at least ultraviolet light transparent at wavelengths between 180 and 315 nm;
one or more light sources configured for illuminating the coiled fluidic pathway of the spiral-shaped tube, wherein the one or more light sources emit light with at least one wavelength within a wavelength range between 180 nm and 315 nm;
wherein the spiral-shaped tube comprises an internal diameter (ID) between 2 mm and 20 mm, a wall thickness (WT) between 5% and 15% of the internal diameter (ID), and an inner coil diameter (ICD) equal to or less than 80 mm;
wherein the spiral-shaped tube comprises a radius of curvature (RC) between 2.0 and 5.0 times the internal diameter (ID), wherein the radius of curvature (RC) of the spiral-shaped tube is calculated according to an equation set forth below:
$$RC = ( ( ICD + ID ) / 2 ) + WT$$
wherein the spiral-shaped tube is geometrically configured for creating a flow of a liquid in the coiled fluidic pathway comprising a Dean number between 300 and 30000.

2. The UV-germicidal irradiation system according to claim 1, wherein the inner coil diameter (ICD) is at least 10 mm.

3. The UV-germicidal irradiation system according to claim 1, wherein the inner coil diameter (ICD) is measured as a distance from inner side to inner side of the spiral-shaped tube after a half turn of the spiral-shaped tube.

4. The UV-germicidal irradiation system according to claim 1, wherein the inner coil diameter (ICD) of the spiral-shaped tube is an average of all inner coil diameters (ICDs) over a length of the spiral-shaped tube.

5. The UV-germicidal irradiation system according to claim 1, wherein the inner coil diameter (ICD) of the spiral-shaped tube comprises a minimum inner coil diameter (ICDmin) representing a smallest inner coil diameter over a length of the spiral-shaped tube and a maximum inner coil diameter (ICDmax) representing a largest inner coil diameter over the length of the spiral-shaped tube, wherein the minimum inner coil diameter (ICDmin) is smaller than the maximum inner coil diameter (ICDmax) by at most 20%.

6. The UV-germicidal irradiation system according to claim 1, wherein the internal diameter (ID) of the spiral-shaped tube is measured from a first point on an inside wall of the spiral-shaped tube to a second point on the inside wall of the spiral-shaped tube, the first point being opposite the second point.

7. The UV-germicidal irradiation system according to claim 1, wherein the internal diameter (ID) of the spiral-shaped tube is an average of all internal diameters (IDs) over a length of the spiral-shaped tube.

8. The UV-germicidal irradiation system according to claim 1, wherein the internal diameter (ID) of the spiral-shaped tube has a minimum internal diameter (IDmin) representing a smallest internal diameter over a length of the spiral-shaped tube and a maximum internal diameter (IDmax) representing a largest internal diameter over the length of the spiral-shaped tube, wherein the minimum internal diameter (IDmin) is smaller than the maximum internal diameter (IDmax) by at the most 10%.

9. The UV-germicidal irradiation system according to claim 1, wherein the polymeric material is selected from amorphous fluoropolymer (AF), polypropylene (PP), fluorinated ethylene propylene (FEP), polyethylene (PE), polytetrafluoroethylene (PTFE), perfluoroalkoxy alkanes (PFA), polyvinylidene fluoride (PVDF), nylon, rubber, latex, acrylic glass, polycarbonate (PC), polystyrene, acrylonitrile butadiene styrene (ABS), a quartz material, or combinations thereof.

10. The UV-germicidal irradiation system according to claim 1, wherein the UV-germicidal irradiation system is configured for creating a flow in the coiled fluidic pathway having a flow speed of the liquid being treated between 0.5 m/s and 10 m/s.

11. The UV-germicidal irradiation system according to claim 1, wherein the spiral-shaped tube comprises a pitch between 1 time an outer tube diameter (OTD) of the spiral-shaped tube and 4 times the outer tube diameter (OTD).

12. The UV-germicidal irradiation system according to claim 1, wherein the internal diameter (ID) is between 2 mm and 18 mm.

13. The UV-germicidal irradiation system according to claim 1, wherein the wall thickness (WT) is between 5% and 15% of the internal diameter (ID).

14. The UV-germicidal irradiation system according to claim 1, wherein the wall thickness (WT) of the spiral-shaped tube is an average of all wall thickness (WTs) over a length of the spiral-shaped tube.

15. The UV-germicidal irradiation system according to claim 1, wherein the wall thickness (WT) of the spiral-shaped tube has a minimum wall thickness (WTmin) representing a smallest wall thickness over a length of the spiral-shaped tube and a maximum wall thickness (WTmax) representing a largest wall thickness over the length of the spiral-shaped tube, where the minimum wall thickness (WTmin) is smaller than the maximum wall thickness (WTmax) by at the most 50%.

16. The UV-germicidal irradiation system according claim 1, wherein the inner coil diameter (ICD) is equal to or less than 70 mm.

17. The UV-germicidal irradiation system according to claim 1, wherein the radius of curvature (RC) is between 2.6 and 4.6 times the internal diameter (ID).

18. The UV-germicidal irradiation system according to claim 1, wherein the spiral-shaped tube is geometrically configured for creating a flow of the fluid in the coiled fluidic pathway having a Dean number between 300 and 25000.

19. The UV-germicidal irradiation system according to claim 1, wherein the UV-germicidal irradiation system further comprises at least one elongated support structure, and wherein the spiral-shaped tube is coiled around the at least one elongated support structure along the longitudinal axis.

20. The UV-germicidal irradiation system according to claim 1, wherein the liquid is an industrial fermentation liquids and/or a fermentation broth.

21. The UV-germicidal irradiation system according to claim 1, wherein the UV-germicidal system further comprises one or more filters, wherein the one or more filters are positioned between the one or more light sources and the coiled fluidic pathway of the spiral shaped tube.

22. (canceled)

23. (canceled)

24. The UV-germicidal system according to claim 1, wherein the UV-germicidal system kills microorganisms that may be in the liquid.

25. A method of germicidal treatment of an industrial fermentation liquid, the method comprising:
providing an industrial fermentation liquid to be treated;
passing the industrial fermentation liquid to be treated through at least one tube in a UV-germicidal irradiation system at a predefined flow speed, the UV-germicidal irradiation system comprising:
at least one tube creating a fluidic pathway, the at least one tube comprising an inlet section, a coil section, and an outlet section, wherein the coil section is a spiral-shaped tube creating a coiled fluidic pathway extending along a longitudinal axis of the spiral-shaped tube from the inlet section to the outlet section, wherein the spiral-shaped tube comprises a polymeric material being at least ultraviolet light transparent at wavelengths between 180 and 315 nm;
one or more light sources configured for illuminating the coiled fluidic pathway of the spiral-shaped tube, wherein the one or more light sources emit light with at least one wavelength within a wavelength range between 180 nm and 315 nm;
wherein the spiral-shaped tube comprises an internal diameter (ID) between 2 mm and 20 mm, a wall thickness (WT) between 5% and 15% of the internal diameter (ID), and an inner coil diameter (ICD) equal to or less than 80 mm;
wherein the spiral-shaped tube comprises a radius of curvature (RC) between 2.0 and 5.0 times the internal diameter (ID), wherein the radius of curvature (RC) of the spiral-shaped tube is calculated according to an equation set forth below:
$$RC = ( ( ICD + ID ) / 2 ) + W T$$
wherein the spiral-shaped tube is geometrically configured for creating a flow of a liquid in the coiled fluidic pathway comprising a Dean number between 300 and 30000;
wherein the method further comprises:
wherein the passing the industrial fermentation liquid to be treated through at least one tube in a UV-germicidal irradiation system is performed while illuminating the coiled fluidic pathway of the spiral-shaped tube with at least one wavelength within a wavelength range between 180 nm and 315 nm from the one or more light sources at a predefined power-output to obtain a germicidal treated fermentation liquid; and
collecting the germicidal treated fermentation liquid.
